SEQUENCE LISTING (83 sequences)

SEQ ID NO: 1 - DNA, 20 bp, Artificial Sequence
  BF105 primer
tcctgctgctatgcctcatc

SEQ ID NO: 2 - DNA, 31 bp, Artificial Sequence
  BR112 primer
tcccttaacttcatgggatatgtcgacggaa

SEQ ID NO: 3 - DNA, 20 bp, Artificial Sequence
  HB-F primer
agtgggcctcagtccgtttc

SEQ ID NO: 4 - DNA, 19 bp, Artificial Sequence
  HB-R primer
tggtattggggccaagtct

SEQ ID NO: 5 - DNA, 20 bp, Artificial Sequence
  HB-F2 primer
ccatcatcttgggctttcgc

SEQ ID NO: 6 - DNA, 25 bp, Artificial Sequence
  HB-R2 primer
taccgctgttaccaattttcttttg

SEQ ID NO: 7 - DNA, 15 bp, Artificial Sequence
  514WF1 probe
tgggctttcgcaaaa

SEQ ID NO: 8 - DNA, 15 bp, Artificial Sequence
  514WF2 probe
tgggcttccgcaaaa

SEQ ID NO: 9 - DNA, 15 bp, Artificial Sequence
  514ML1 probe
tgggcttacgcaaaa

SEQ ID NO: 10 - DNA, 15 bp, Artificial Sequence
  514ML2 probe
tgggcttgcgcaaaa

SEQ ID NO: 11 - DNA, 15 bp, Artificial Sequence
  514ML3 probe
tgggccttcgcaaaa

SEQ ID NO: 12 - DNA, 15 bp, Artificial Sequence
  514ML4 probe
tgggcctccgcaaaa

SEQ ID NO: 13 - DNA, 15 bp, Artificial Sequence
  514ML5 probe
tgggcctacgcaaaa

SEQ ID NO: 14 - DNA, 15 bp, Artificial Sequence
  514ML6 probe
tgggcctaggcaaaa

SEQ ID NO: 15 - DNA, 15 bp, Artificial Sequence
  528WL1 probe
gtttctcctggctca

SEQ ID NO: 16 - DNA, 15 bp, Artificial Sequence
  528WL2 probe
gtttctcttggctca

SEQ ID NO: 17 - DNA, 15 bp, Artificial Sequence
  528MM probe
gtttctcatggctca

SEQ ID NO: 18 - DNA, 15 bp, Artificial Sequence
  529WA1 probe
tctcttggctcagtt

SEQ ID NO: 19 - DNA, 15 bp, Artificial Sequence
  529WA2 probe
tctcttggcccagtt

SEQ ID NO: 20 - DNA, 15 bp, Artificial Sequence
  529WA3 probe
tctcttggcacagtt

SEQ ID NO: 21 - DNA, 15 bp, Artificial Sequence
  529WA4 probe
tctcttggcgcagtt

SEQ ID NO: 22 - DNA, 15 bp, Artificial Sequence
  529MT1 probe
tctcttgactcagtt

SEQ ID NO: 23 - DNA, 15 bp, Artificial Sequence
  529MT2 probe
tctcttgacccagtt

SEQ ID NO: 24 - DNA, 15 bp, Artificial Sequence
  529MT3 probe
tctcttgacacagtt

SEQ ID NO: 25 - DNA, 15 bp, Artificial Sequence
  529MT4 probe
tctcttgccgcagtt

SEQ ID NO: 26 - DNA, 15 bp, Artificial Sequence
  548WA1 probe
tgtctggctttcagt

SEQ ID NO: 27 - DNA, 15 bp, Artificial Sequence
  548WA2 probe
tgtctggccttcagt

SEQ ID NO: 28 - DNA, 15 bp, Artificial Sequence
  548WA3 probe
tgtctggcattcagt

SEQ ID NO: 29 - DNA, 15 bp, Artificial Sequence
  548WA4 probe
tgtctggcgttcagt

SEQ ID NO: 30 - DNA, 15 bp, Artificial Sequence
  548MV1 probe
tgtctggttttcagt

SEQ ID NO: 31 - DNA, 15 bp, Artificial Sequence
  548MV2 probe
tgtctggtcttcagt

SEQ ID NO: 32 - DNA, 15 bp, Artificial Sequence
  548MV3 probe
tgtctggtattcagt

SEQ ID NO: 33 - DNA, 15 bp, Artificial Sequence
  548MV4 probe
tgtctggtgttcagt

SEQ ID NO: 34 - DNA, 18 bp, Artificial Sequence
  YMDD probe
tcagttatatggatgatg

SEQ ID NO: 35 - DNA, 18 bp, Artificial Sequence
  YVDD probe
tcagttatgtggatgatg

SEQ ID NO: 36 - DNA, 17 bp, Artificial Sequence
  YIDD1 probe
cagttatatagatgatg

SEQ ID NO: 37 - DNA, 17 bp, Artificial Sequence
  YIDD2 probe
cagttatatcgatgatg

SEQ ID NO: 38 - DNA, 17 bp, Artificial Sequence
  YIDD3 probe
cagttatattgatgatg

SEQ ID NO: 39 - DNA, 17 bp, Artificial Sequence
  YSDD1 probe
cagttatagtgatgatg

SEQ ID NO: 40 - DNA, 17 bp, Artificial Sequence
  YSDD2 probe
cagttatagcgatgatg

SEQ ID NO: 41 - DNA, 17 bp, Artificial Sequence
  YSDD3 probe
cagttattctgatgatg

SEQ ID NO: 42 - DNA, 17 bp, Artificial Sequence
  YSDD4 probe
cagttattccgatgatg

SEQ ID NO: 43 - DNA, 17 bp, Artificial Sequence
  YSDD5 probe
cagttattcagatgatg

SEQ ID NO: 44 - DNA, 17 bp, Artificial Sequence
  YSDD6 probe
cagttattcggatgatg

SEQ ID NO: 45 - DNA, 17 bp, Artificial Sequence
  555WV probe
gatgatgtggtattggg

SEQ ID NO: 46 - DNA, 17 bp, Artificial Sequence
  555MI1 probe
gatgatattgtattggg

SEQ ID NO: 47 - DNA, 17 bp, Artificial Sequence
  555MI2 probe
gatgatatagtattggg

SEQ ID NO: 48 - DNA, 15 bp, Artificial Sequence
  528N-C probe
gtttctcgtggctca

SEQ ID NO: 49 - DNA, 15 bp, Artificial Sequence
  528N-I-T probe
gtttctctctggctc

SEQ ID NO: 50 - DNA, 15 bp, Artificial Sequence
  528N-I-A probe
gtttctcactggctc

SEQ ID NO: 51 - DNA, 15 bp, Artificial Sequence
  528N-I-G probe
gtttctcgctggctc

SEQ ID NO: 52 - DNA, 15 bp, Artificial Sequence
  528N-I-C probe
gtttctccctggctc

SEQ ID NO: 53 - DNA, 15 bp, Artificial Sequence
  528N-D-1 probe
gtttctctggctcag

SEQ ID NO: 54 - DNA, 15 bp, Artificial Sequence
  528N-D-2 probe
cgtttcttggctcag

SEQ ID NO: 55 - DNA, 17 bp, Artificial Sequence
  552N-C-C probe
tcagttatctggatgat

SEQ ID NO: 56 - DNA, 17 bp, Artificial Sequence
  552N-C-T probe
tcagttatttggatgat

SEQ ID NO: 57 - DNA, 17 bp, Artificial Sequence
  552N-I-A probe
agttatatgagatgatg

SEQ ID NO: 58 - DNA, 17 bp, Artificial Sequence
  552N-I-C probe
agttatatgcagatgat

SEQ ID NO: 59 - DNA, 17 bp, Artificial Sequence
  552N-I-G probe
agttatatggagatgat

SEQ ID NO: 60 - DNA, 17 bp, Artificial Sequence
  552N-I-T probe
agttatatgtagatgat

SEQ ID NO: 61 - DNA, 17 bp, Artificial Sequence
  552N-I-AG probe
gttatatgagagatgat

SEQ ID NO: 62 - DNA, 17 bp, Artificial Sequence
  552N-I-TC probe
gttatatgtcagatgat

SEQ ID NO: 63 - DNA, 17 bp, Artificial Sequence
  552N-D-1 probe
tcagttattggatgatg

SEQ ID NO: 64 - DNA, 17 bp, Artificial Sequence
  552N-D-2 probe
tcagttatggatgatga

SEQ ID NO: 65 - DNA, 17 bp, Artificial Sequence
  552N-D-3 probe
tcagttatatgatgatg

SEQ ID NO: 66 - DNA, 17 bp, Artificial Sequence
  552N-D-4 probe
tcagttatatatgatga

SEQ ID NO: 67 - DNA, 17 bp, Artificial Sequence
  552N-D-5 probe
tcagttatagatgatga

SEQ ID NO: 68 - DNA, 17 bp, Artificial Sequence
  555N-C-TC probe
gatgatttcgtattggg

SEQ ID NO: 69 - DNA, 17 bp, Artificial Sequence
  555N-C-CC probe
gatgatctcgtattggg

SEQ ID NO: 70 - DNA, 17 bp, Artificial Sequence
  555N-I-A probe
gatgatgtaggtattgg

SEQ ID NO: 71 - DNA, 17 bp, Artificial Sequence
  555N-I-T probe
gatgatgttggtattgg

SEQ ID NO: 72 - DNA, 17 bp, Artificial Sequence
  555N-I-G probe
gatgatgtgggtattgg

SEQ ID NO: 73 - DNA, 17 bp, Artificial Sequence
  555N-I-C probe
gatgatgtcggtattgg

SEQ ID NO: 74 - DNA, 17 bp, Artificial Sequence
  555N-I-AC probe
atgatgtacggtattgg

SEQ ID NO: 75 - DNA, 17 bp, Artificial Sequence
  555N-I-TC probe
atgatgttcggtattgg

SEQ ID NO: 76 - DNA, 17 bp, Artificial Sequence
  555N-I-GC probe
atgatgtgcggtattgg

SEQ ID NO: 77 - DNA, 17 bp, Artificial Sequence
  555N-I-AT probe
atgatgtatggtattgg

SEQ ID NO: 78 - DNA, 17 bp, Artificial Sequence
  555N-I-GG probe
atgatgtggggtattgg

SEQ ID NO: 79 - DNA, 17 bp, Artificial Sequence
  555N-D-1 probe
agatgatgggtattggg

SEQ ID NO: 80 - DNA, 17 bp, Artificial Sequence
  555N-D-2 probe
agatgatgtgtattggg

SEQ ID NO: 81 - DNA, 17 bp, Artificial Sequence
  555N-D-3 probe
agatgattggtattggg

SEQ ID NO: 82 - DNA, 17 bp, Artificial Sequence
  555N-D-4 probe
agatgatggtattgggg

SEQ ID NO: 83 - DNA, 17 bp, Artificial Sequence
  555N-D-5 probe
gagatgatgtattgggg

(19) United States
(12) Patent Application Publication
(10) Pub. No.: US 2006/0134792 A1
(43) Pub. Date: Jun. 22, 2006

(54) Microarray comprising probes for drug-resistant hepatitis b virus detection, quality control and negative control, and method for detecting drug-resistant hepatitis b virus using the same

(75) Inventors: Cheol-Min Kim, Busan-city (KR); Hee-Kyung Park, Seoul (KR); Mong Cho, Busan-city (KR); Hyun-Jung Jang, Busan-city (KR); Jeong Heo, Busan-city (KR)

Correspondence Address: CLARK & ELBING LLP, 101 FEDERAL STREET, BOSTON, MA, 02110, US

(73) Assignee: Genein Co., Ltd., Busan-city (KR)

(21) Appl. No.: 10/567,072

(22) PCT Filed: Aug. 2, 2004
(86) PCT No.: PCT/KR04/01940

§ 371 Date: Feb. 3, 2006

(30) Foreign Application Priority Data
Aug. 5, 2003 (KR) 10-2003-0054204

(51) Int. Cl. C12Q 1/00

(52) U.S. Cl. 436/6

(57) ABSTRACT

Provided are a microarray manufactured using a mixture of target probes for drug-resistant HBV detection, quality control probes for controlling quality in probe hybridization and fabrication of microarrays, and negative control probes for determining the presence and ratio of more than one type, i.e., a wild type and a mutant in a codon, measuring a background of non-specific cross-hybridization, and discriminating homozygotes and heterozygotes, and a method of detecting a drug-resistant HBV, controlling the quality of a microarray, determining the presence and ratio of more than one type, and determining positive and false positive probes at the same time using the microarray. The microarray, which includes the target probes for drug-resistant HBV detection, the QC probes, and the negative control probes, can detect a drug-resistant HBV, control quality in fabrication of microarrays and hybridization, determine the presence and ratio of more than one type, i.e., a wild type and a mutant, determine positive and false positive probes by measuring a background of non-specific cross-hybridization, and discriminate homozygotes and heterozygotes. When a plurality of sets of probes, each set containing target probes, QC probes, and negative control probes, are immobilized on a support of the microarray, detection of resistance in HBV to multiple drugs, quality control, and determination as to the presence and ratio of a wild type and a mutant and whether each probe is positive or false positive can be rapidly and accurately performed.

### TECHNICAL FIELD

The present invention relates to a microarray for detecting a drug-resistant hepatitis B virus (hereinafter, referred to as “HBV”). More particularly, the present invention relates to a microarray comprising target probes for detection of drug-resistant HBV, quality control (QC) probes for quality control of microarray fabrication and hybridization, and negative control probes for determining the presence and ratio of one or more wild-types and mutants and detecting positive and false positive probes by measurement of a background of nonspecific cross hybridization attached to a support, and a HBV detection method and a HBV diagnostic kit using the same.

### BACKGROUND ART

It is estimated that 5-6% of the Korean adult population and about 5% of the global population, i.e., 350 million of the global population, are chronic hepatitis B virus carriers [Chutima Pramoolsinsup. J Gastroenterol Hepatol, 17: S125-S145 (2002)]. The ultimate treatment of hepatitis B is to suppress proliferation of HBVs, thereby preventing liver damage and thus the progress to hepatocirrhosis or liver cancer.

There have been many studies of immunosuppression and antiviral therapies for chronic hepatitis B, and the like. In the 1980s, interferon was introduced as a treatment of chronic hepatitis B. However, there arose problems such as cost ineffectiveness, parenteral administration, drug resistance, side effects, and recurrence after treatment. In view of these problems, lamivudine (3-TC) was approve to be safe and efficient for the treatment of hepatitis B [Seong Gyu Hwang, Korean Journal of Hepatology, 8: 93-100 (2002)].

Lamivudine ((−)-β-L-2′,3′-dideoxy-3′-thiacytidine), which is a nucleoside analogue, mainly provides two mechanisms against HBV proliferation. According to a first mechanism, lamivudine inhibits the activities of both DNA-dependent polymerase activity and RNA-dependent polymerase (reverse transcriptase) activity of HBV DNA polymerase. That is, lamivudine prevents HBV proliferation by inhibiting the synthesis of minus (−)-strand DNA from pregenomic RNA by reverse transcriptase and the synthesis of plus (+)-strand DNA from minus-strand DNA by DNA polymerase. According to a second mechanism, lamivudine serves as a chain terminator to prevent the elongation of HBV DNA. The proliferation of HBV can be prevented by the combination of the two mechanisms.

It is reported that as the duration of lamivudine administration increases, the negative conversion rate of HBV DNA, ALT (alanine aminotransferase) normalization, and HBeAg seroconversion rate increase. It is also reported that long-term lamivudine therapy for HBeAg negative, anti-HBe positive, and high serum HBV DNA concentration pre-core mutant (10-15% of HBVs in Korea) provides excellent biochemical and virological enhancement effects. However, 12-month and 24-month cumulative recurrence rates after administration of lamivudien are as high as 37.5% and 49.2%, respectively [Song B C, Suh D J, Lee H C, Chung Y H, and Lee Y S. Hepatology, 32: 803-806, 2000]. For this reason, long-term (more than one year, generally) administration of lamivudine is required. The long-term administration of lamivudine can cause HBV mutations associated with lamivudine resistance in patients with chronic hepatitis B, resulting in continued HBV proliferation. Long-term use of famciclovir, which is another nucleoside analogue, can also cause continued proliferation of a famciclovir resistant virus.

Recently, it has been determined that adefovir, which is a new antiviral nucleoside analogue, prevents lamivudine- and famciclovir-resistant viruses fro emerging due to the long-term use of these drugs. In this respect, it is reported that adefovir is a solution to problems associated with lamivudine- and famciclovir-resistant viruses [Seong Gyu Hwang, Korean Journal of Hepatology, 8: 93-100 (2002)]. Early diagnosis of drug-resistance in patients with chronic hepatitis B is very important for individual-specific treatment, for example, to determine whether various drugs can be prescribed together to a patient. Therefore, an early diagnosis technique and an accurate, quick drug-resistance determination technique are required for effective treatment of HBV drug-resistance.

Conventional HBV detection methods can be used only for HBV DNA detection. Enzyme immunoassay (EIA) and radioimmunoassay (RIA) for HBV DNA detection are relatively simple because automatic systems are used therefor but are not sensitive enough to detect mutant HBVs in resistance tests. A PCR technique is known as a highly sensitive method but cannot be used to detect point-mutant HBV.

Generally, viral resistance in antiviral therapy is defined by the increased viral DNA levels in serum during therapy (also defined as phenotypic resistance) and the selection of a mutation in a viral polymerase gene that is not detectable in major viral species prior to therapy and not found in the consensus sequences derived from data banks (also defined as genotypic resistance). However, a more detailed definition of the phenotypic resistance requires in vitro tissue culture, especially when identifying a new mutation. For the definition of the genotypic resistance, up to now, as the standard for the clinical definition of virus resistance, i.e., detection and diagnosis of virus resistance, studies of the sequence-based molecular assays of viral polymerase genes such as direct sequencing of PCR products (Ling et al., 1996), restriction fragment length polymorphism (RFLP) analysis of PCR products (Chayama et al., 1998 and Allen et al., 1999), a line probe assay (Stuyver et al., 2000), and a clonal analysis (Seigners and Stuyver et al., 2000) have been performed. Hitherto, however, there have been no reports of a virus resistance assay using a DNA chip or an oligonucleotide chip that can detect single nucleotide sequence variations and can detect various mutations in only one experiment [Fabien Zoulim. J Clin Virol., 21: 243-253, 2001].

The term “microarray” refers to a biochip including biomolecules such as DNA and proteins immobilized at a high density on a microsized substrate made of glass, silicon, or nylon. By assaying a binding pattern between target materials of interest to be assayed and the immobilized biomolecules, i.e., probes, genes or proteins associated with a specific disease can be detected. Microarrays can be referred to as either DNA chips if DNA is immobilized or protein chips if proteins are immobilized. DNA chips can be classified into pin microarray chips, inkjet chips, photolithography chips, and electronic array chips according to a method of immobilizing DNA on a surface of the DNA chip.

A microarray assay involves spotting target probes onto a support using a spotter, hybridizing target materials to the target probes, and scanning for analysis. In the fabrication of microarrays, quality control (referred to as “QC” hereinafter) of microarray elements is very important. In particular, the immobilization of target probes is a critical factor that determines the quality of microarrays. In this respect, to obtain highly reliable results from experiments and diagnoses using microarrays, there is a need to check the quality of the microarrays, especially to determine whether the immobilization of target probes is successful, prior to hybridization. In the present invention, QC in immobilization and hybridization in the fabrication of microarrays can be achieved by immobilizing both target probes and reference oligonucleotide QC probes on a support.

Drug resistance in HBV may be induced from a single wild type or mutant alone, a combination of a wild type and a mutant, or a combination of different types of mutants. It is very difficult to determine the presence and ratio of more than one type in the combinations of different types.

However, for more accurate diagnosis, the presence and ratio of more than one type must be determined and positive and false positive probes must be discriminated by measuring a background of non-specific cross-hybridization. In the present invention, to this end, negative control probes artificially modified not to contain all of the sequences of target probes are used. By measuring a background of non-specific cross-hybridization between target products and the negative control probes, which do not match the sequences of the target products, the presence and ratio of more than one type can be determined and positive and false positive probes can be discriminated.

To detect drug resistance to lamivudine and famciclovir, the inventors have developed an oligonucleotide chip including target probes that specifically react with the drug-resistant HBV. Using the oligonucleotide chip, instead of conventional serological methods for HBV detection, drug-resistant mutants occurring due to, for example, sequence substitution, in addition to the presence of HBVs, can be rapidly and accurately detected. In order to prevent drawbacks and inefficiency in quality control using conventional microarrays, a mixture of target probes for drug-resistant HBV detection and fluorescently-labeled QC probes is spotted onto a support of the microarray. In order to determine the presence and ratio of more than one type and discriminate positive and false positive probes by measuring a background of non-specific cross-hybridization, a mixture of negative control probes, which are sequence modified from the target probes for homozygotes and heterozygotes discrimination, and target probes is spotted onto a support of the microarray. Using the microarray according to the present invention, it is possible to detect a drug-resistant HBV, check the qualities of the individual probes immobilized on a slide prior to hybridization, determine the presence and ratio of more than one type, i.e., a wide type and a mutant, discriminate positive and false positive probes, and discriminate homozygotes and heterozygotes. This method of using the microarray is economical, rapid, and accurate.

### BRIEF DESCRIPTION OF THE DRAWINGS

FIG. 1A illustrates a microarray according to an embodiment of the present invention, in which a) illustrates a plurality of sets of spots on a support of the microarray, each set containing target probes for drug-resistant HBV detection and quality control (QC) probes, b) illustrates a set of probes among the sets of a) immobilized on the microarray, and c) illustrates the types of the probes of b) immobilized on the microarray;

FIG. 1B illustrates a microarray according to another embodiment of the present is invention, in which a) illustrates a plurality of sets of spots on a support of the microarray, each set containing target probes for drug-resistant HBV detection, QC probes, and negative control probes for determining the presence and ratio of more than one type of HBV and for measuring a background of non-specific cross-hybridization, b) illustrates a set of probes among the sets of a) immobilized on the microarray, and c) illustrates the types of the probes of b) immobilized on the microarray;

FIG. 2 illustrates QC probes for verifying the immobilization and hybridization of target probes;

FIGS. 3A through 3C illustrate the results of scanning a slide before washing after the immobilization of a mixture of QC probes and target probes in a predetermined ratio as in FIG. 1A;

FIGS. 3D through 3E illustrate the results of scanning a slide before washing after the immobilization of a mixture of QC probes, target probes, and negative control probes in a predetermined ratio as in FIG. 1B;

FIG. 4 illustrates the result of scanning a slide before washing after the immobilization of only target probes for drug-resistant HBV detection without QC probes;

FIGS. 5A, 5B, and 5C illustrate the results of hybridization specific to probes for drug-resistant HBV detection when using a susceptible wild type HBV (FIG. 5A), a drug-resistant HBV with YVDD mutation at codon 552 (FIG. 5B), and a drug-resistant HBV with YIDDD3 mutation at codon 552;

FIGS. 6A, 6B, and 6C illustrate the results of hybridization using viruses including more than two types, in particular, wild type YMDD and mutant YVDD at codon 552 (FIG. 6A), mutant 528MM at codon 528 and mutant YVDD at codon 552 (FIG. 6B), and mutant 528MM at codon 528 and mutants YVDD and YVDD3 at codon 552 (FIG. 6C);

FIGS. 7A, 7B, and 7C comparatively illustrate the results of nucleotide sequencing (FIGS. 7A and 7B) and an array using the microarray according to the present invention (FIG. 7C) using the viruses of FIG. 6C; and

FIGS. 8A through 8D illustrate the results of non-specific cross-hybridization background measurements using negative control probes, in which FIGS. 8A and 8B illustrate the results of scanning the microarray of FIG. 1B, and FIGS. 8C and 8D illustrate the results of hybridization using microarrays for drug-resistant HBV detection.

### DETAILED DESCRIPTION OF THE INVENTION

### Technical Goal of the Invention

The present invention provides a microarray for the rapid and accurate diagnosis of drug resistant-HBV.

The present invention also provides a microarray including fluorescently labeled QC probes that is capable of detecting and diagnosing a drug-resistant HBV based on only one experiment and quality controlling probe immobilization and hybridization.

The present invention also provides a microarray including negative control probes that is capable of determining the presence and the ratio of one or more wild types and mutants, discriminating a positive probe that matches a target sequence and a false positive probe that does not match the target sequence by measuring a background of non-specific cross-hybridization, discriminating homozygotes and heterozygotes, and/or genotyping.

The present invention also provides a method of simultaneously detecting resistance in HBV to multiple drugs using the microarray, controlling the quality of the microarray, determining the presence and the ratio of more than one type and discriminating positive and false positive probes to target products.

### Disclosure of the Invention

According to an aspect of the present invention, there is provided a microarray including target probes immobilized on a support for detecting drug-resistance HBV.

In the specification, the target probes mean any probes that can be specifically bind to target products, such as target genes, associated with HBV drug resistance in a sample.

In the microarray of the present invention, the support may be any support commonly used in the fabrication of microarrays, such as a slide glass, a membrane, a semiconductor chip, a silicon, and a gel, but are not limited thereto.

In the microarray of the present invention, the target probes may be any biomaterial capable of detecting a HBV and can be selected according to the type of the microarray. Preferably, the target probes are selected from cDNAs, oligonucleotides, DNA analogues, such as peptide nucleic acids (PNAs), locked nucleic acids (LNAs) and hexitol nucleic acids (HNAs), peptides, and proteins.

In the microarray of the present invention, the target probes for drug-resistant HBV detection may be oligonucleotides that can specifically bind to a target gene inducing resistance to lamivudine and/or famciclovir. Preferably, the target probes may be oligonucleotides including the nucleotide sequences of point mutations at codons 528, 529, and 514 in domain B and at codons 552, 548, and 555 in domain C HBV DNA polymerase gene that induce resistance to lamivudine, and/or oligonucleotides including the nucleotide sequences of point mutations at codons 528 and 529 in domain B and at codon 555 in domain C of the HBV DNA polymerase gene that induce resistance to famciclovir. More preferably, the target probes are at least one kind of oligonucleotides having SEQ ID NOs. 7 through 47.

The microarray of the present invention may further comprise negative control probes for detecting the presence and ratio of more than one type, detecting positive and false positive probes by measuring a background of non-specific cross-hybridization, discriminating homozygotes and heterozygotes, and/or genotyping.

The negative control probes may be prepared by substituting, inserting, or deleting at least one nucleotide sequence among the nucleotide sequences of the target probes not to be hybridized with a target product. Preferably, the negative control probes comprise at least one kind of oligonucleotides having SEQ ID NOs. 48 through 83.

In the microarray according to the present invention, QC probes labeled with a fluorescent material having a different excitation/emission wavelength from a fluorescent material used to label the target product and target probes may be included in each spot.

In the microarray according to the present invention, the QC probes may be oligonucletides having the same sequences as the target probes that have at least one nucleotide labeled with a fluorescent material, or arbitrary sequences that have at least one nucleotide labeled with a fluorescent material.

In the microarray according to the present invention, the fluorescent material used to label the quality control probes may be at least one selected from the group consisting of Pyrene, Cyanine 2, GFP, Calcein, FITC, Alexa 488, FAM, Fluorescein Chlorotriazinyl, Fluorescein, Rhodamine 110, Oregon Green, Magnesium Green, Calcium Green, JOE, Cyanine 3, Tetramethylrhodamine, TRITC, TAMRA, Rhodamine Phalloidin, Pyronin Y, Lissamine, ROX, Calcium Crimson, Texas Red, Nile Red, Cyanine 5, and Thiadicarbocyanine.

The microarray according to the present invention may further comprise negative control probes for detecting the presence and ratio of more than one type, detecting positive and false positive probes by measuring a background of non-specific cross-hybridization, discriminating homozygotes and heterozygotes, and/or genotyping in addition to the QC probes. The negative control probes and QC probes may be included in each spot.

According to another aspect of the present invention, there is provided use of the above-described microarray to simultaneously perform at least one process selected from the group consisting of detecting a drug-resistant HBV, quality controlling probe immobilization and hybridization, detecting the presence and ratio of more than one type, detecting positive and false positive probes by measuring a background of non-specific cross-hybridization, discriminating homozygotes and heterozygotes, and genotyping.

According to another aspect of the present invention, there is provided a HBV diagnostic kit comprising the above-described microarray according to the present invention.

According to another aspect of the present invention, there is provided a primer or probe for detecting HBV drug resistance, the primer or probe comprising one of nucleotide sequences having SEQ ID NOs. 1 through 47.

According to another aspect of the present invention, there is provided a negative control probe for detecting the presence and ratio of more than one type, detecting positive and false positive probes by measuring a background of non-specific cross-hybridization, discriminating homozygotes and heterozygotes, and/or genotyping, the negative control probe being prepared by substituting, inserting, or deleting at least one nucleotide sequence among the nucleotide sequences of the target probes for detecting HBV drug resistance that have one of nucleotide sequences of SEQ ID NOs. 7 through 47. For example, the negative control probe according to the present invention may have one of nucleotide sequences of SEQ ID NOs. 48 through 83.

Throughout the specification, the sequences of oligonucleotides are described with reference to only one strand, primers or probes can be designed using any one of DNA double strands of target products. Therefore, both sense and antisense nucleotide sequences having the above-described SEQ ID NOs. lie within the scope of the present invention.

Hereinafter, the present invention will be described in more detail.

The present invention provides a microarray with target probes for HBV drug resistance immobilized on a support. The present invention also provides a method of fabricating a microarray by immobilizing a mixture of fluorescently-labeled QC probes or negative control probes and target probes for drug-resistant HBV detection in a predetermined ratio or a mixture of negative control probes, QC probes and target probes in a predetermined ratio on a support. The present invention provides a microarray fabricated using the method that includes fluorescently-labeled QC probes and negative control probes.

In the microarray according to the present invention, the target probes for drug-resistant HBV detection and fluorescently-labeled QC probes or negative control probes are included in each spot on the support. The microarray may include a plurality of sets of spots on a support of the microarray, each set containing target probes, fluorescently-labeled QC probes and negative control probes for each target codon. A plurality of samples can be simultaneously tested for drug-resistant HBV detection and diagnosis using the microarray based on only one experiment. In addition, using the microarray according to the present invention, it is possible to accurately and rapidly quality control the immobilization and hybridization of every probe in the manufacture of a microarray, to detect the presence and ratio of more than one type, and to discriminate positive and false positive probes to target products by measuring a background of non-specific cross-hybridization.

In the present invention, instead of using the QC probes separately, some of the target probes may be labeled with a different fluorescent material and used as QC probes. In this case, probe immobilization and hybridization between target products and target probes can be verified using only one kind of probes. The fluorescent material used to label the QC probes has a different excitation/emission wavelength from the fluorescent material used to label the target products. By analyzing the QC probes on the microarray, which are labeled with a fluorescent material emitting light of a predetermined wavelength, prior to an assay for diagnostic or research purposes, the immobilization of target probes can be verified. In addition, the hybridization to the target probes immobilized on the support can be verified using the QC probes that do not cause spectral interference.

In the present invention, any fluorescent material having a predetermined emission wavelength, which differs from the emission wavelength of a fluorescent material for target products, can be used to label the QC probes. For example, when Cy5 is used as a fluorescent material for detecting the coupling between the target products and target probes, Cy3 or TAMRA, which are fluorescent materials having different emission wavelengths from Cy5, can be used to label the QC probes. In the present invention, like target probes immobilized on a support, the QC probes may be oligonucleotides having nucleotide sequences that are complementary to the target products or arbitrary nucleotide sequences. At least one nucleotide of the oligonucleotide used as the QC probes can be labeled with the fluorescent material. Any region of the probe, such as 3′-terminal, 5′-terminal, or a middle region, can be labeled with the fluorescent material. A spacer may be interposed or not between the nucleotide sequence of the QC probes and the fluorescent material. The spacer can be any biomolecules that can link the fluorescent materials and the probes without affecting hybridization. Examples of the spacer include C-3 linker, C-6 linker, C-6 TFA linker, C-5 amino modifier, C-12 linker, Amino dT C2 linker, Amino dT C6 linker, 3′ branched amino CPGs, 3′ C3 amino modifier, 3′ C7 amino modifier, 5′ Thiol C-2 linker, 5′ Thiol C-6 linker, 5′ Thiol C-6 S—S, 3′ Thiol C3, etc. In addition, another spacer may be interposed between the QC spacers and the support. This spacer can be any molecules that can link the probes and the support without affecting hybridization. Examples of the spacer that can be used for this purpose include C-3 linker, C-6 linker, C-6 TFA linker, C-5 amino modifier, C-12 linker, Amino dT C2 linker, Amino dT C6 linker, 3′ branched amino CPGs, 3′ C3 amino modifier, 3′ C7 amino modifier, 5′ Thiol C-2 linker, 5′ Thiol C-6 linker, 5′ Thiol C-6 S—S, 3′ Thiol C3, etc.

In the microarray according to the present invention, the QC probes and target probes are included in each spot. The microarray according to the present invention may include other kinds of target probes according to the purpose of use. Using the above-described QC probes, whether probes have been immobilized or not, the status of the immobilized probes, such as spot pattern and concentration, etc., and the hybridization of target products can be verified.

In the microarray according to the present invention, the target probes and negative control probes are included in each spot. In the present invention, in addition to the target probes having nucleotide sequences with which a wild type and a mutant in a codon of a target gene can be detected, negative control probes are constructed by modifying at least one nucleotide of the nucleotide sequence of each of the target probes using a method such as substitution, insertion, deletion, etc. not to be hybridized with target products. The negative control probes can be biological materials such as cDNA, oligonucleotides, PNA, peptides, proteins, etc., which are selected according to the type of the microarray.

In the present invention, the target probes for drug-resistant HBV detection can be oligonucleotides having nucleotide sequences that can specifically bind to a drug-resistant target gene, such as target nucleotide sequences that induce resistance to, for example, lamivudine, famciclovir, etc. Preferred examples of oligonucleotides for the target probes include an oligonucleotide including the nucleotide sequence of a HBV DNA polymerase gene with point mutations at codons 552, 548 and 555 in YMDD motif of domain C and at codons 528, 529 and 514 in domain B that induce resistance to lamivudine, and an oligonucleotide including the nucleotide sequence of a HBV DNA polymerase gene with point mutations at codons 528 and 529 in domain B and at codon 555 in domain C that induce resistance to famciclovir. More preferred examples of oligonucleotides for the target probes include oligonucleotides of SEQ ID NOs. 7 through 47 (for lamivudine resistance detection), oligonucleotides of SEQ ID NOs. 15 through 25 (for famciclovir resistance detection), and oligonucleotides of SEQ ID NOs. 45 through 47 (for famciclovir resistance detection).

In most lamividine-resistant variants, YMDD motif (tyrosine-methionine-aspartate-aspartate) in domain C of the HBV DNA polymerase gene is changed to YVDD (M52V) with valine substituting methionine at codon 552 or YIDD (M552I) with isoleucine substituting methionine at codon 552. Due to such a change of the bases, lamivudine cannot suppress the function of the HBV DNA polymersase any longer. The substitution of methionine by isoleucine or valine leads to shorter side chains and reduces the binding affinity of lamivudine by changing a binding pocket therefor, thereby suppressing the function of the HBV DNA polymerase. HBV resistance is also known to occur from a mutation (L528M) with methionine substituting leucine at codon 528 in conversed domain B of the polymerase gene. Accordingly, lamivudine-resistance related mutations are roughly classified into either group I with double mutations in domains B and C (L528M and M552V) or group II with a single mutation in domain C (M552I) (Nafa S, Ahmed S, Tavan D, Pichoud C, Berby F, and Stuyver L, et al. Hepatology, 32: 1078-1088, 2000, Fabien Zoulim. J Clin Virol., 21: 243-253, 2001). Codon 528 in domain B and codon 555 in domain C of the HBV DNA polymerase gene are associated with resistance to famciclovir, which is another nucleoside analogue (Xiong X, Yang H, Westland C E, Zou R, and Gibbs C S. Hepatology. 31: 219-224, 2000, Anna S. F. Lok, Fabien Zoulim, Stephen Locarnini, and Alessandra angia, et al. J Clin Microbiol., 40: 3729-3734, 2002). In addition to codons 552 and 528, codons 514, 529 and 548 are related with resistance to lamivudine and famciclovir. In addition to valine and isoleucine, serine at codon 552 is known to be associated with a mutation inducing resistance to lamivudine (Karl P. Fischer, Klaus S. Guffreund, and D. Lorne Tyrrell. Drug Resist Updat. 4: 118-128, 2001, Chau-Ting Yeh, Rong-Nan Chien, Chia-Ming Chu, and Yun-Jan Liaw. Hepatology. 31: 1318-1326, 2000, Hubert G. M. Niesters, Robert A. de Man, and Suzan D. Pas, et al. J. Med. Microbiol. 51: 659-699, 2002).

Using the microarray according to the present invention, drug resistance induced by a point mutation in a limited domain can be easily detected by DNA hybridization or reverse hybridization (Rossau R., Traore H., De Beenhouwer H., Mijs W., Jannes G., De Rijk P., Portaels F. Antimicrob Agents Chemother, 41: 2093-2098, 1997). When using reverse hybridization, a drug-resistant mutant can be detected in a virus including both a wild type and a mutant earlier than using a sequencing method, and the wild type and the mutant can be identified (Anna S. F. Lok, Fabien Zoulim, Stephen Locarnini, and Alessandra Mangia, et al. J Clin Microbiol., 40: 3729-3734, 2002).

A method of detecting HVB drug resistance according to the present invention is based on the detection of a mutation in sequences that response to drugs. A microarray used in the method is manufactured using a pin or inkjet microarrayer, which is commonly used in the field, by immobilizing probes designed to detect a wile type and a mutant in a target nucleotide sequence of an antiviral drug onto a solid support. The detection method according to the present invention allows simultaneous detection of various mutations using only one experiment and is more rapid, accurate and convenient than conventional costly drug-resistance detection methods requiring skillful technicians, thereby enabling earlier effective HBV treatment.

The present invention also provides a method of detecting HBV drug resistance using a microarray and simultaneously controlling the quality of the microarray and a method of determining the presence and ratio of more than one type and discriminating positive and false positive probes to target products. In particular, a mixture of target probes and QC probes is immobilized on a support such that each spot contains both target and QC probes and hybridized with target products. By detecting whether the target products have coupled with the probes, it is possible to detect a drug-resistant HBV and simultaneously control the quality of spotting and hybridization on the microarray. It is also possible to determine the presence and ratio of more than one type, for example, a wild type and a mutant, in a codon and to discriminate positive and false positive probes from non-specific cross-hybridization.

The present invention also provides a HBV diagnostic kit including the microarray. In addition to the microarray, the diagnostic kit according to the present invention may further includes a hybridization reaction solution, a PCR kit containing primers for amplifying target products, a unhybridized-DNA washing solution, a cover slip, a dye, a undyed product washing solution, a user manual, etc.

The present invention will be described in greater detail with reference to the following examples. The following examples are for illustrative purposes and are not intended to limit the scope of the invention.

### Effect of the Invention

As described above, the present invention provides an assay method using a microarray in which target probes for HBV drug resistance detection, QC probes labeled with a fluorescent material, and negative control probes for detecting the presence of more than one type and discriminating positive and false positive probes are immobilized on a support such that each spot contains the target probes and QC probes or negative control probes, and a HBV diagnostic kit using the microarray. Use of the microarray according to the present invention allows easy, accurate control of the quality of hybridization and target probes immobilized on the support, detection of resistance to drugs, such as lamivudine, famciclovir, etc., and discrimination of positive and false positive probes by measurement of a background of non-specific cross-hybridization, all based on only one experiment. Since each spot on the microarray contains both the target probes and QC probes, whether the target probes have been hybridized, the status of the target probes, such as pattern, concentration, etc., and factors affecting hybridization can be detected to control the quality of the microarray while detecting resistance to drugs, such as lamivudine, famciclovir, etc. In addition, since the QC probes are mixed with the negative control probes, whether the negative control probes have been immobilized, the status of the negative control probes, such as pattern, concentration, etc., the presence and ratio of more than one type, such as a wild type and a mutant, in a codon, can be detected, and positive and false positive probes can be discriminated by measuring a background of non-specific cross-hybridization. The negative control probes can be used in other microorganisms, in addition for the detection of a wild type and a mutant in HBV, and for genotyping and discriminating homozygotes and heterozygotes. In addition, since a plurality of sets of probes are immobilized on a support, HBV drug resistance can be detected using multiple samples based on only one experiment, thereby reducing the diagnosis time and cost compared to existing commercialized methods. Both a wild type and a mutant cannot be simultaneously detected using conventional methods such as sequencing, whereas even more than two mutants can be detected using the microarray according to the present invention with high sensitivity. The rapid, accurate, convenient detection of mutants enables earlier drug resistance detection and effective HBV treatment.

### EMBODIMENTS

### EXAMPLE 1

### HBV DNA Isolation

A blood sample taken from a HBV carrier was stored in a refrigerator for 1 hour for coagulation and subjected to centrifugation at 3000 rpm for 5 minutes to separate serum. The separated serum was stored at −70° C., and 200 μL of HBV DNA was extracted from the serum using a QIAmp DNA Blood Mini Kit (QIAGEN Inc., CA, USA) and used as a template DNA for polymerase chain reaction (PCR).

### EXAMPLE 2

### HBV Detection and Preparation of Target Probes for Drug-Resistant HBV Detection

Oligonucleotide probes and primers used in the present invention were prepared by synthesizing probes each including 15-25 nucleotides with a dT spacer of a length of C6-15 at 5′-terminal (5′-Amino-Modifier C6-15) using a Perkin Elmer DNA synthesizer (USA) and isolating by PAGE. The prepared target probes and primers are listed in Table 1 below.

In Table 1, SEQ ID NOs. 1 through 6 are forward and reverse primers for HBV DNA polymerase gene, SEQ ID NOs. 1 and 2 are outer primers for primary PCR, and SEQ ID NOs. 3 through 6 are biotin-labeled inner primers. SEQ ID NOs. 7 through 14 are probes for lamivudine detection, SEQ ID NOs. 7 and 8 are probes for detecting a wild type at codon 514, and SEQ ID NOs. 9 through 14 are probes for detecting a mutant at codon 514. SEQ ID NOs. 15 through 25 and NOs. 45 through 47 are probes for detecting lamivudine and famciclovir, SEQ ID NOs. 15 and 16 are probes for detecting a wild type at codon 528, SEQ ID NO. 17 is a probe for detecting a mutant at codon 528, SEQ ID NOs. 18 through 21 are probes for detecting a wild type at codon 529, SEQ ID NOs. 22 through 25 are probes for detecting a mutant at codon 529, SEQ ID NOs. 45 and 46 are probes for detecting a wild type at codon 555, SEQ ID NO. 47 is a probe for detecting a mutant at codon 555, SEQ ID NOs. 26 through 44 are probes for detecting lamivudine, SEQ ID NOs. 25 through 29 are probes for detecting a wild type at codon 548, SEQ ID NOs. 30 through 33 are probes for detecting a mutant at codon 548, SEQ ID NO. 34 is a probe for detecting a wild type at codon 552, and SEQ ID NOs. 35 through 44 are probes for detecting a mutant at codon 552.

TABLE 1
Target probes and primers for drug-resistant HBV detection

| | | | |
|---|---|---|---|
| CODON | Primer or | | SEQ ID |
| (Antiviral Drug) | Probe Name | Nucleotide Sequences | NOs |

| | | | |
|---|---|---|---|
| Primers | BF105 | TCCTGCTGCTATGCCTCATC | 1 |
| | BR112 | TCCCTTAACTTCATGGGATATGTDGADGGAA | 2 |
| | HB-F | 5′-biotin-AGTGGGCCTCAGTCDGTTTC-3′ | 3 |
| | HB-R | 5′-biotin-TGGTATTGGGGDCAAGTCT-3′ | 4 |
| | HB-F2 | 5′-biotin-CCATCATCTTGGGCTTTDGC-3′ | 5 |
| | HH-R2 | 5′-biotin-TACCGCTGTTACCAATTTTCTTTTG-3′ | 6 |
| 514 | 514WF1 | 5′-T₁₆-TGGGCTTTCGCAAAA-3′ | 7 |
| (lamivudine) | 514WF2 | 5′-T₁₆-TGGGCTTDCGCAAAA-3′ | 8 |
| | 514ML1 | 5′-T₁₆-TGGGCTTACGCAAAA-3′ | 9 |
| | 514ML22 | 5′-T₁₆-TGGGCTTGCGCAAAA-3′ | 10 |
| | 514ML3 | 5′-T₁₆-TGGGCCTTCGCAAAA-3′ | 11 |
| | 514ML4 | 5′-T₁₆-TGGGCCTCCGCAAAA-3′ | 12 |
| | 514ML5 | 5′-T₁₆-TGGGCCTACGCAAAA-3′ | 13 |
| | 514ML6 | 5′-T₁₆-TGGGCCTAGGCAAAA-3′ | 14 |
| 528 | 528L1 | 5′-T₁₆-GTTTCTCCTGGCTCA-3′ | 15 |
| (lamivudine & | 528WL2 | 5′-T₁₆-GTTTCTCTTGGCTCA-3′ | 16 |
| famciclovir) | 528MM | 5′-T₁₆-GTTTCTCATGGCTCA-3′ | 17 |
| 529 | 529WA1 | 5′-T₁₆-TCTCTTGGCTCAGTT-3′ | 18 |
| (lamivudine & | 529WA2 | 5′-T₁₆-TCTCTTGGCCCAGTT-3′ | 19 |
| famciclovir) | 529WA3 | 5′-T₁₆-TCTCTTGGCACAGTT-3′ | 20 |
| | 529WA4 | 5′-T₁₆-TCTCTTGGCGCAGTT-3′ | 21 |
| | 529MT1 | 5′-T₁₆-TCTCTTGACTCAGTT-3′ | 22 |
| | 529MT2 | 5′-T₁₆-TCTCTTGACCCAGTT-3′ | 23 |
| | 529MT3 | 5′-T₁₆-TCTCTTGACACAGTT-3′ | 24 |
| | 529MT4 | 5′-T₁₆-TCTCTTGDCGCAGTT-3′ | 25 |
| 545 | 548WA1 | 5′-T₁₆-TGTCTGGCTTTCAGT-3′ | 26 |
| (lamivudine) | 548WA2 | 5′-T₁₆-TGTCTGGDCTTCAGT-3′ | 27 |
| | 548WA3 | 5′-T₁₆-TGTCTGGCATTCAGT-3′ | 28 |
| | 548WA4 | 5′-T₁₆-TGTCTGGDGTTCAGT-3′ | 29 |
| | 548MV1 | 5′-T₁₆-TGTCTGGTTTTCAGT-3′ | 30 |
| | 548MV2 | 5′-T₁₆-TGTCTGGTCTTCAGT-3′ | 31 |
| | 548MV3 | 5′-T₁₆-TGTCTGGTATTCAGT-3′ | 32 |
| | 548MV4 | 5′-T₁₆-TGTCTGGTGTTCAGT-3′ | 33 |
| 552 | YMDD | 5′-T₁₆-TCAGTTATATGGATGATG-3′ | 34 |
| (lamivudine) | YVDD | 5′-T₁₆-TCAGTTATGTGGATGATG-3′ | 35 |
| | YIDD1 | 5′-T₁₆-CAGTTATATAGATGATG-3′ | 36 |
| | YIDD2 | 5′-T₁₆-CAGTTATATCGATGATG-3′ | 37 |
| | YIDD3 | 5′-T₁₆-CAGTTATATTGATGATG-3′ | 38 |
| | YSDD1 | 5′-T₁₆-CAGTTATAGTGATGATG-3′ | 39 |
| | YSDD2 | 5′-T₁₆-CAGTTATAGCGATGATG-3′ | 40 |
| | YSDD3 | 5′-T₁₆-CAGTTATTCTGATGATG-3′ | 41 |
| | YSDD4 | 5′-T₁₆-CAGTTATTDCGATGATG-3′ | 42 |
| | YSDD5 | 5′-T₁₆-CAGTTATTCAGATGATG-3′ | 43 |
| | YSDD6 | 5′-T₁₆-CAGTTATTDGGATGATG-3′ | 44 |
| 555 | 555WV | 5′-T₁₆-GATGATGTGGTATTGGG-3′ | 45 |
| (lamivudine & | 555MI1 | 5′-T₁₆-GATGATATTGTATTGGG-3′ | 46 |
| famciclovir) | 555MI2 | 5′-T₁₆-GATGATATAGTATTGGG-3′ | 47 |

### EXAMPLE 3

### Preparation of Fluorescent Dye-Labeled QC Probes

A fluorescent material having an emission wavelength different from a fluorescent material used for target probes is selected to label QC probes. For example, when Cy5 is used with an emission filter for 670 nm to detect the binding of target products and target probes on an oligonucleotide chip, Cy3 or TAMRA, which have emission wavelengths near 570 nm, can be used when synthesizing QC probes. When both Cy3 and Cy5 are used to label target probes on a cDNA chip, a fluorescent material having a different emission wavelength from Cy3 and Cy5 is used when synthesizing QC probes.

In the present invention, the fluorescent material used to label QC probes includes, but is not limited to, at least one of materials listed in Table 2 that have different emission wavelengths from a fluorescent material used to label target products.

TABLE 2
Fluorescent materials that can be used in microarray for QC

| | Excitation | Emission | Emission |
|---|---|---|---|
| Fluorescent materials | (nm) | (nm) | filter |
| Pyrene | 340 | 376, 395 | 430 |
| Cyanine 2 | 489 | 506 | 508 |
| GFP | 488 | 507 | 508 |
| Calcein | 494 | 517 | 522 |
| FITC | 494 | 518 | 522 |
| Alexa 4B8 | 490 | 520 | 522 |
| FAM | 490 | 520 | 522 |
| Fluorescein | 492 | 514 | 522 |
| Chlorotriazinyl | | | |
| Fluorescein | 494 | 517 | 522 |
| Rhodamine 110 | 500 | 525 | 522 |
| Oregon Green | 500 | 524 | 522 |
| Magnesium Green | 506 | 531 | 530 |
| Calcium Green | 506 | 533 | 530 |
| JOE | 524 | 550 | 549 |
| Cyanine 3 | 550 | 570 | 570 |
| Tetramethylrhodamine | 550 | 570 | 570 |
| TRITC | 547 | 572 | 570 |
| TAMRA | 560 | 582 | 578 |
| Rhodamine Phalloidin | 550 | 575 | 578 |
| Pyronin Y | 555 | 580 | 578 |
| Lissamine | 570 | 590 | 592 |
| ROX | 588 | 608 | 614 |
| Calcium Crimson | 590 | 615 | 614 |
| Texas Red | 595 | 615 | 614 |
| Nile Red | 549 | 628 | 630 |
| Cyanine 5 | 649 | 670 | 670 |
| Thiadicarbocyanine | 651 | 671 | 670 |

In this example, Cy5 was used as a fluorescent material for target probes and TMARA was used as a fluorescent material for QC probes expressed as 5′-Amino-Modifier C6 20-50 mer-TAMRA QC probes. The QC probes used in the present invention have the following sequences.

5′-TTT TTT TTT TTT TTT TTT TTT TTT TTT TTT-
TAMRA-3′
5′-TTT TTT TTT TTT TTT Tgg Tgg ggT gTg gTg TTT gA-
TAMRA-3′

As shown in FIG. 2, the QC probes used in the present invention, which have the same sequence as the target probes or an arbitrary sequence and immobilized on a support with a spacer, may be directly labeled with the fluorescent material or with a spacer between the nucleotides and the fluorescent material.

### EXAMPLE 4

### Preparation of Negative Control Probes

Negative control probes used in the present invention were prepared in the same manner as for the target probes but to have nucleotide sequences different from the target probes. The nucleotide sequences of the negative control probes were designed by modifying the nucleotide sequences of the target probes for each codon by substitution, insertion, deletion, etc. The negative control probes used in the present invention have SEQ ID NOs. 18 through 53 in Table 4 below. The negative control probes in Table 4 are listed for exemplary purpose and not limited thereto, and thus can be modified further by substitution, insertion, deletion, etc., in at least one nucleotide sequence.

In Table 3, SEQ ID NOs. 48 through 54 are negative control probes for codon 528, SEQ ID NOs. 55 through 67 are negative control probes for codon 552, and SEQ ID NOs. 68 through 83 are negative control probes for codon 555.

TABLE 3
Negative control probes

| CODON | | | SEQ ID |
|---|---|---|---|
| (Modifications) | Probe Name | Nucleotide Sequeuces | NOs |
| 528 (Sub) | 528N-C | 5′-T₁₆-GTTTCTCGTGGCTCA-3′ | 48 |
| 528 (Ins) | 528N-I-T | 5′-T₁₆-GTTTCTCTCTGGCTC-3′ | 49 |
| | 528N-I-A | 5′-T₁₆-GTTTCTCACTGGCTC-3′ | 50 |
| | 528N-I-G | 5′-T₁₆-GTTTCTCGCTGGCTC-3′ | 51 |
| | 528N-I-C | 5′-T₁₆-GTTTCTCCCTGGCTC-3′ | 52 |
| 528 (Del) | 528N-D-1 | 5′-T₁₆-GTTTCTCTGGCTCAG-3′ | 53 |
| | 528N-D-2 | 5′-T₁₆-DGTTTCTTGGCTCAG-3′ | 54 |
| 552 (Sub) | 552N-C-C | 5′-T₁₆-TCAGTTATCTGGATGAT-3′ | 55 |
| | 552N-C-T | 5′-T₁₆-TCAGTTATTTGGATGAT-3′ | 56 |
| 552 (Ins) | 552N-I-A | 5′-T₁₆-AGTTATATGAGATGATG-3′ | 57 |
| | 552N-I-C | 5′-T₁₆-AGTTATATGCAGATGAT-3′ | 58 |
| | 552N-I-G | 5′-T₁₆-AGTTATATGGAGATGAT-3′ | 59 |
| | 552N-I-T | 5′-T₁₆-AGTTATATGTAGATGAT-3′ | 60 |
| | 552N-I-AG | 5′-T₁₆-GTTATATGAGAGATGAT-3′ | 61 |
| | 552N-I-TC | 5′-T₁₆-GTTATATGTCAGATGAT-3′ | 62 |
| 552 (Del) | 552N-D-1 | 5′-T₁₆-TCAGTTATTGGATGATG-3′ | 63 |
| | 552N-D-2 | 5′-T₁₆-TCAGTTATGGATGATGA-3′ | 64 |
| | 552N-D-3 | 5′-T₁₆-TCAGTTATATGATGATG-3′ | 65 |
| | 552N-D-4 | 5′-T₁₆-TCAGTTATATATGATGA-3′ | 66 |
| | 552N-D-5 | 5′-T₁₆-TCAGTTATAGATGATGA-3′ | 67 |
| 555 (Sub) | 555N-C-TC | 5′-T₁₆-GATGATTTCGTATTGGG-3′ | 68 |
| | 555N-C-CC | 5′-T₁₆-GATGATCTCGTATTGGG-3′ | 69 |
| 555 (Ins) | 555N-I-A | 5′-T₁₆-GATGATGTAGGTATTGG-3′ | 70 |
| | 555N-I-T | 5′-T₁₆-GATGATGTTGGTATTGG-3′ | 71 |
| | 555N-I-G | 5′-T₁₆-GATGATGTGGGTATTGG-3′ | 72 |
| | 555N-I-C | 5′-T₁₆-GATGATGTCGGTATTGG-3′ | 73 |
| | 555N-I-AC | 5′-T₁₆-ATGATGTACGGTATTGG-3′ | 74 |
| | 555N-I-TC | 5′-T₁₆-ATGATGTTCGGTATTGG-3′ | 75 |
| | 555N-I-GC | 5′-T₁₆-ATGATGTGCGGTATTGG-3′ | 76 |
| | 555N-I-AT | 5′-T₁₆-ATGATGTATGGTATTGG-3′ | 77 |
| | 555N-I-GG | 5′-T₁₆-ATGATGTGGGGTATTGG-3′ | 78 |
| 555 (Del) | 555N-D-1 | 5′-T₁₆-AGATGATGGGTATTGGG-3′ | 79 |
| | 555N-D-2 | 5′-T₁₆-AGATGATGTGTATTGGG-3′ | 80 |
| | 555N-D-3 | 5′-T₁₆-AGATGATTGGTATTGGG-3′ | 81 |
| | 555N-D-4 | 5′-T₁₆-AGATGATGGTATTGGGG-3′ | 82 |
| | 555N-D-5 | 5′-T₁₆-GAGATGATGTATTGGGG-3′ | 83 |

### EXAMPLE 5

### Probe Immobilization on Support

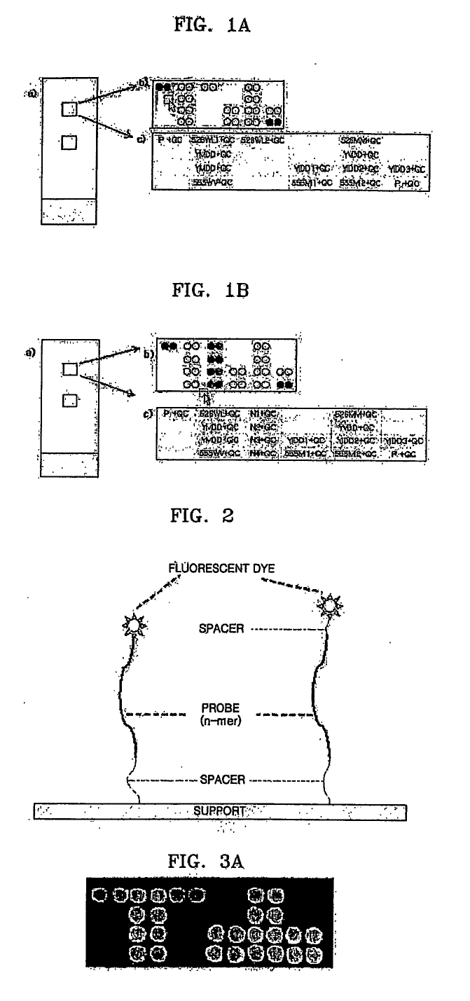

Each of the target probes prepared in Example 2 and the negative control probes prepared in Example 4 was diluted to a concentration of 30˜50 pmol and transferred to positions of a 96-well microplate illustrated in b) and c) of FIG. 1. 1˜5 pmol of the QC probes prepared in Example 3 and a micro-spotting solution or a 3×SSC solution were added to each of the wells and mixed. Although 1 pmol of the QC probes and 50 pmol of the negative control probes were used in this example, the ratio of mixing these probes can be varied within a range in which the QC probes do not affect detected results. The mixture of the probes was spotted onto a slide glass (membrane) used as a substrate using a microarrayer (Cartesian Technologies, PLXSYS 7500 SQLX Microarrayer, U.S.A.). Two spots of each kind of the probes were attached to the support and left in a slide box at room temperature for 24 hours or in a dry oven at 50° C. for about 5 hours to be fixed to the surface of the support.

### EXAMPLE 6

### Preparation of Target Products

To amplify target products for drug-resistant HBV detection, forward and reverse primers labeled with biotin in Example 2 were used. Amplification was performed up to about 200 bp to include codon 514 in domain B and codon 555 in domain C of the HBV polymerase gene of the DNA extracted in Example 1. In this example, primary PCR was carried out using primers BF105 and BR112 and secondary PCR was carried out using primers HB-F2 and HB-R2.

In particular, 4 μl of the HBV DNA separated in Example 1 was mixed with primers BF105 and BR112 to obtain 25 μl of a PCR solution. After the PCR solution was reacted at 94° C. for 4 minutes for sufficient denaturation, 30 cycles of amplification at 94° C. for 1 minute, at 58° C. for 1 minute, and at 72° C. for 1 minute were carried out and followed by a single final extension at 72° C. for 10 minutes. Secondary PCR was carried out using 2 μl of the primary PCR products and biotin-labeled HB-F2 and HB-R2 under the same conditions as for the primary PCR.

### EXAMPLE 7

### Assessment of Probe Immobilization Quality

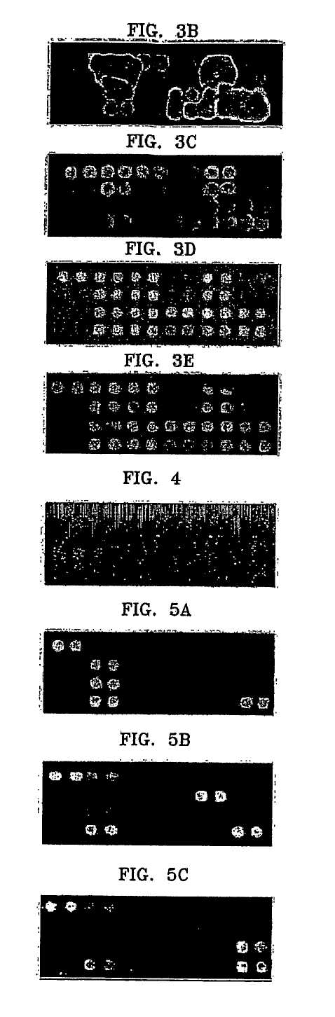

The degree of immobilization of the probes on the slide and the status of the immobilized probes before washing were investigated. After the immobilization of the probes on the microarray in Example 5, whether the probes had been successfully immobilized on the slide and the status of spots of the probes were analyzed using a laser scanner. FIGS. 3A through 3E show the scanned results, and FIG. 4 shows the result of scanning a microarray after probe immobilization and before washing, which spots on a support, each set containing target probes for drug-resistant HBV detection and QC probes, b) illustrates a set of probes among the sets of a) immobilized on the microarray, c) illustrates the types of the probes of b) immobilized on the microarray.

In FIG. 1B, a) illustrates a microarray with a plurality of sets of spots on a support, each set containing target probes for drug-resistant HBV detection, QC probes, and negative control probes for determining the presence and ratio of more than one type of HBV and for non-specific cross-hybridization background measurement, b) illustrates a set of probes among the sets of a) immobilized on the microarray, and c) illustrates the types of the probes of b) immobilized on the microarray.

In FIGS. 1A and 1B, “P” denotes a positive control probe, “QC” denotes a quality control probe, “N1” through “N4” denote negative control probes for codons, and “528WL” denotes the fact that probes 528WL1 and 528WL2 are mixed. Due to positional difference between point mutations YVDD and YIDD at codon 552, wild type (YMDD) probes having a difference (mer) in length were constructed. Wild type probes 528WL1 and 528WL2 for codon 528 were immobilized on the same position. Negative control probes were constructed for individual codons and point mutations YVDD and YIDD at codon 552. This exemplary layout of the probes can be varied.

FIG. 2 illustrates QC probes labeled with a fluorescent dye that can be used to detect the immobilization and hybridization of the probes. In particular, one of the QC probes, which are spaced above a support by a spacer, has the nucleotide sequence of a target probe or an arbitrary nucleotide sequence directly labeled with the fluorescent dye, and the other QC probe has a spacer between the nucleotide sequence and the fluorescent dye. The nucleotide sequence of the QC probes (n-mer) labeled with the fluorescent dye is the same as the nucleotide sequence of the target probe or an arbitrary nucleotide sequence.

FIGS. 3A through 3E show the results of scanning slide glasses before washing after the immobilization of a mixture of QC probes and target probes in a predetermined ratio or a mixture of target probes, QC probes and negative control probes in a predetermined ratio to determine whether the probes have been successfully immobilized or the status of the immobilized probes. The shapes or sizes of the spots are almost the same but the sizes of the spots may vary depending on the type of the support. FIG. 3A illustrates that the shape and concentration of probes immobilized on the slide are excellent. FIG. 3B illustrates a case where a plurality of probes was manufactured in the same manner as in Example 5 except that no QC probes were used.

### EXAMPLE 8

### Unimmobilized Probe Washing

The slide glass after the process in Example 5 or 8 was washed with a 0.2% SDS buffer solution and then distilled water at room temperature to remove unimmobilized probes. The washed slide glass was immersed in a sodium borohydride (NaBH₄) solution for 5 minutes and then washed again at 100° C. Final washing with a 0.1% SDS solution and then distilled water was followed by centrifugation to fully dry the slide glass.

### EXAMPLE 9

### Hybridization and Staining

The biotin-labeled target products prepared in Example 6 were thermally treated to be denaturated into single strands and cooled to 4° C. A hybridization reaction solution containing 1˜5 μl of the target products was mixed with a dye (20 SSPE 3 μl, 22.2M Formamide 1.35 μl, Bovine serum albumin 0.5 μl, Salmon sperm DNA 0.1 μl, Cy5-streptavidin (Amersharm Pharmacia Biotech, U.S.A.) 0.06 μl) to obtain 10 μl of a reaction solution. This hybridization reaction solution was portioned on the slide glass after the process in Example 8, and the slide glass was covered with a cover slop to block light and reacted at 40° C. for 30 minutes.

### EXAMPLE 10

### Unhybridized Target Product Washing

To wash out unhybridized target products, the cover slip was removed using a 2×SSC washing solution (300 mm NaCl, 30 mm Na-Citrate, pH 7.0), and the slide was washed with 2×SSC and then 0.2×SSC, followed by centrifugation to fully dry the slide glass.

### EXAMPLE 11

### Result Analysis

The hybridized result was scanned using a non-confocal laser scanner (GenePix 4000A, Axon Instruments, U.S.A.) and analyzed by image analysis.

FIGS. 1A and 1B illustrate microarrays according to embodiments of the present invention. In particular, in FIG. 1A, a) illustrates a microarray with a plurality of sets of conglomerate due to a problem in an immobilization process so that the shapes and concentrations of the immobilized probes are irregular. FIG. 3C illustrates a case where the shapes and concentrations of the immobilized probes are irregular and some probes are not immobilized on the slide. In the cases of FIGS. 3B and 3C, final experimental results are greatly influenced so that the concentration and shape of the probes immobilized on each of the slides are varied after the immobilization is complete. FIGS. 3D through 3E illustrate the results of scanning slide glasses before washing after the immobilization of a mixture of target probes and negative control probes in a predetermined ratio. In FIG. 3D, all the probes are optimally immobilized on the slide glass. In FIG. 3E, some of the probes have irregular shapes and concentrations.

FIG. 4 illustrates the results of scanning a slide glass before washing after the immobilization of only target probes for drug-resistant HBV detection without QC probes. Unlike the results in FIGS. 3A through 3E, since no QC probe has not been immobilized, no information can be obtained from each of the spots.

FIGS. 5A, 5B, and 5C illustrate the results of hybridization specific to probes for drug-resistant HBV detection. FIG. 5A illustrates the result of hybridization using a drug-susceptible wild type HBV. In this case, hybridization occurred in positive control probes, 528WL1, YMDD, and 555WV among the target probes in c) of FIG. 1A. FIG. 5B illustrates the result of hybridization using a mutant with valine (GTG) substituting methionine (ATG) at codon 552. In this case, hybridization occurred in positive control probes, 528WL1, YVDD, and 555WV among the target probes in c) of FIG. 1A. FIG. 5C illustrates the result of hybridization using a mutant with ATT (YIDD3), which is one of three kinds of isoleucines ATA(YIDD1), ATC (YIDD2), and ATT (YIDD3) substituting methionine at codon 552. In this case, hybridization occurred in positive control probes, 528WL1, YIDD3, and 555WV among the target probes in c) of FIG. 1A. As is apparent from the results, using a microarray according to the present invention, susceptibility to drug can be identified, and different types of mutants can be rapidly and accurately detected.

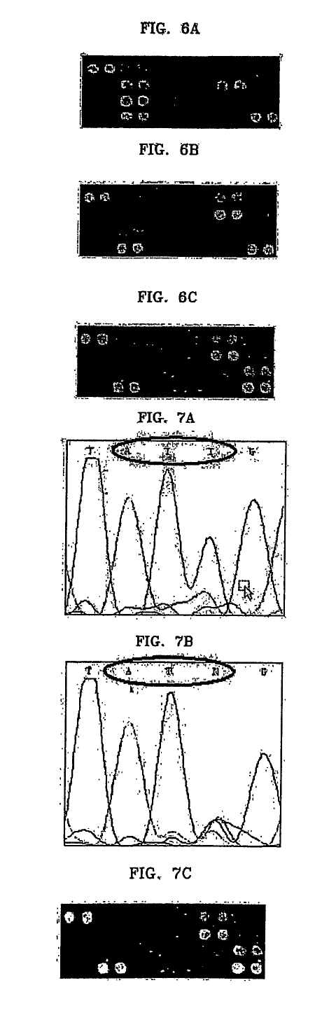

FIGS. 6A, 6B, and 6C illustrate the results of hybridization using viruses including more than two types. FIG. 6B illustrates the detected result of hybridization using a virus including a wide type YMDD and a mutant YVDD at codon 552. In this case, positive control probes, 528WL1, and 555WV among the target probes in c) of FIG. 1A were detected. Hybridization reaction occurred in both YMDD and YVDD at codon 552. FIG. 6B illustrates the detected result of hybridization using a virus including a mutant at codon 528 and a mutant at codon 552. In this case, positive control probes, 528MM, YVDD, and 555MV among the target probes in c) of FIG. 1A were detected. FIG. 6C illustrates the detected result of hybridization using a virus including three types of mutants, 528MM at codon 528 and YVDD and YIDD3 at codon 552. In this case, positive control probes, 528MM, YVDD, YIDD3, and 555WV among the target probes in c) of FIG. 1A were detected. As is apparent from the results of FIGS. 6A through 6C, using a microarray according to the present invention, a wild type and a mutant or different types of mutants in the same codon, as well as a single type in a codon as illustrated in FIGS. 5A through 5C, can be accurately detected.

FIGS. 7A and 7B illustrate the results of nucleotide sequencing when mutants YVDD and YIDD3 exist at codon 552 as in FIG. 6C. In FIG. 7A, only YIDD3 between the two types appears. In FIG. 7B, point mutation sites at which nucleotide sequences cannot be detected are denoted by “N”. FIG. 7C shows the result of an assay using the microarray according to the present invention when two types of mutants exist. The mutation of the used HBV DNA was experimentally identified to be restriction fragment length polymorphism including YVDD and YIDD3. As is apparent from the results of FIGS. 6A through 6C, when two or more types of mutants exit, the types of the mutants can be accurately detected using the microarray according to the present invention. Most HBVs include two or more types of genes therein, and thus it is important to accurately detect all the types of the genes for treatment with anti-viral drugs lamivudine and famciclovir. The microarray according to the present invention is very useful for this purpose.

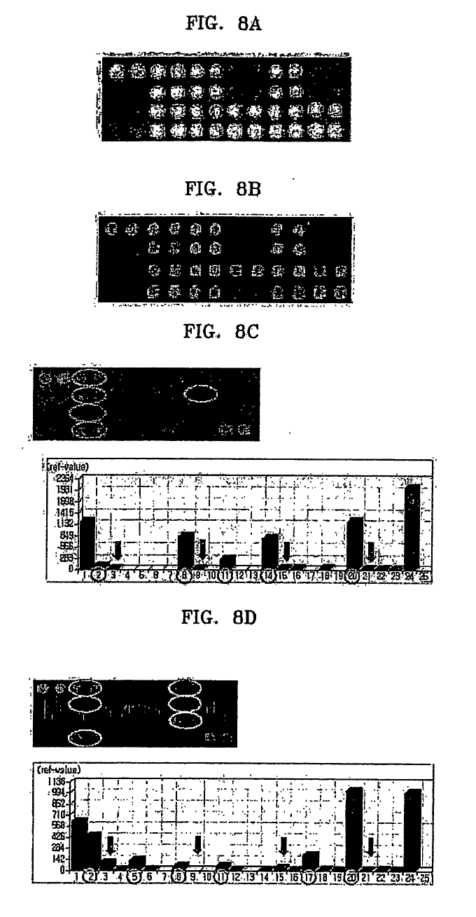

FIGS. 8A through 8D illustrate the results of non-specific cross-hybridization background measurements using negative control probes. FIGS. 8A and 8B illustrate the results of scanning the microarray of FIG. 1B. In FIG. 8A, probes attached to a non-spot region appear, and the concentrations of the attached probes are inconstant. In FIG. 8B, no 555M2 probe appears because that probe has been removed from the microarray in spotting and washing processes. FIGS. 8C and 8D illustrate the results of hybridization using microarrays for drug-resistant HBV detection. Since the presence and ratio of a wild type and a mutant, or more than one mutant at a single codon cannot be identified from the scanned images, quantitative fluorescence data of each of the probes immobilized on the micoarrays for drug-resistive HBV detection are also provided. The levels of fluorescence from the probes can be read from normalized fluorescent signals. The presence and ratio of more than one type in each codon can be identified from the levels of fluorescence of negative control probes. It also can be determined whether each probe is positive or false positive.

It is apparent from FIG. 8C that a wild type exists at codon 518 and mutants YMDD and YVDD exist at codon 552. The mixed type cannot be detected in the image. FIG. 8D shows that a mixed type (wild type and mutant) exits at codon 528 and YMDD (wild type) and YVDD and YIDD2 (mutants) exit at codon 552. When determining whether probes are positive or false positive, the probe is determined to be positive if the fluorescence of each target probe is greater than the fluorescence of probe 3, which is a negative control probe NI for codon 528), probe 9, which is a negative control probe N2 for YVDD, probe 15, which is a negative control probe N3 for YIDD, and probe 21, which is a negative control probe N4 for codon 555. Otherwise, the probe is determined to be negative.

Using negative control probes on a microarray according to the present invention, a background of non-specific hybridization can be measured to discriminate positive probes, which react with target products, and false positive probes involved in non-specific hybridization. In addition, positive probes can be accurately detected when only one or more than one type exists in a single codon. Furthermore, homozygotes and heterozygotes can be discriminated and genotyped based on the method and principles of detecting a wild type and mutants in a codon.

1. A microarray with target probes for detecting drug-resistant HBV on a support, wherein the target probes comprise oligonucleotides including the nucleotide sequences of point mutations at codons 528, 529, and 514 in domain B and at codons 552, 548, and 555 in domain C of a HBV DNA polymerase gene that induce resistance to lamivudine and/or oligonucleotides including the nucleotide sequences of point mutations at codons 528 and 529 in domain B and at codon 555 in domain C of the HBV DNA polymerase gene that induce resistance to famciclovir.

2. The microarray of claim 1, wherein the support is a slide glass, a membrane, a semiconductive chip, a silicon, or a gel.

3. The microarray of claim 1, wherein the target probes are cDNA, oligonucleotides, DNA analogues, peptides, or proteins.

4. (canceled)

5. (canceled)

6. The microarray of claim 1, wherein the target probes comprise at least one kind of oligonucleotides including the nucleotide sequences of SEQ ID NOs. 7 through 47.

7. The microarray of claim 1, further comprising negative control probes for detecting the presence and ratio of more than one type, detecting positive and false positive probes by measuring a background of non-specific cross-hybridization, discriminating homozygotes and heterozygotes, and/or genotyping.

8. The microarray of claim 7, wherein the negative control probes are prepared by substituting, inserting, or deleting at least one nucleotide sequence among the nucleotide sequences of the target probes not to be hybridized with a target product.

9. The microarray of claim 8, wherein the negative control probes comprise at least one kind of oligonucleotides including the nucleotide sequences of SEQ ID NOs. 48 through 83.

10. The microarray of any one of claims 1 through 6, wherein claim lfurther comprising quality control probes labeled with a fluorescent material having a different excitation/emission wavelength from a fluorescent material used to label the target product and target probes are included in each spot.

11. The microarray of claim 10, wherein the quality control probes are oligonucletides having the same sequences as the target probes that have at least one nucleotide labeled with a fluorescent material, or arbitrary sequences that have at least one nucleotide labeled with a fluorescent material.

12. The microarray of claim 10, wherein the fluorescent material used to label the quality control probes is at least one selected from the group consisting of Pyrene, Cyanine 2, GFP, Calcein, FITC, Alexa 488, FAM, Fluorescein Chlorotriazinyl, Fluorescein, Rhodamine 110, Oregon Green, Magnesium Green, Calcium Green, JOE, Cyanine 3, Tetramethylrhodamine, TRITC, TAMRA, Rhodamine Phalloidin, Pyronin Y, Lissamine, ROX, Calcium Crimson, Texas Red, Nile Red, Cyanine 5, and Thiadicarbocyanine.

13. (canceled)

14. Method for simultaneously performing at least one process selected from the group consisting of detecting a drug-resistant HBV, quality controlling probe immobilization and hybridization, detecting the presence and ratio of more than one type, detecting positive and false positive probes by measuring a background of non-specific cross-hybridization, discriminating homozygotes and heterozygotes, and genotyping using the microarray of any one of claims 1 through 3 and 6 through 12.

15. (canceled)

16. A primer or probe for detecting HBV drug resistance, the primer or probe comprising one of nucleotide sequences having SEQ ID NOs. 1 through 47.

17. A negative control probe for detecting the presence and ratio of more than one type, detecting positive and false positive probes by measuring a background of non-specific cross-hybridization, discriminating homozygotes and heterozygotes, and/or genotyping, the negative control probe being prepared by substituting, inserting, or deleting at least one nucleotide sequence among the nucleotide sequences of the target probes for detecting drug-resistant HBV that have one of nucleotide sequences of SEQ ID NOs. 7 through 47.

18. The negative control probe of claim 17 having one of nucleotide sequences of SEQ ID NOs. 48 through 83.
